(19) United States
(12) Patent Application Publication
(10) Pub. No.: US 2023/0128956 A1
(43) Pub. Date: Apr. 27, 2023

(54) MYOCARDIAL MULTIPARAMETRIC ULTRASOUND IMAGING METHOD AND SYSTEM

(71) Applicant: Xi'an Jiaotong University, Xi'an (CN)

(72) Inventors: Diya WANG, Xi'an (CN); Mingxi WAN, Xi'an (CN); Pengfei XU, Xi'an (CN); Xiaoke ZHANG, Xi'an (CN); Qiang SU, Xi'an (CN)

(21) Appl. No.: 18/145,896

(22) Filed: Dec. 23, 2022

(30) Foreign Application Priority Data
Dec. 31, 2021 (CN) 202111677331.5
Dec. 31, 2021 (CN) 202111677333.4

(51) Int. Cl. A61B 8/08; A61B 8/00

(52) U.S. Cl. CPC A61B 8/0883; A61B 8/488; A61B 8/5207; A61B 8/4488; A61B 8/485

(57) ABSTRACT

A myocardial multi-parametric ultrasound imaging method includes: emitting alternately a plurality of ascending and descending large-acoustic domain diverging waves to myocardial tissue to obtain raw channel data; performing beamforming to obtain a plurality of radio frequency data; performing myocardial edge detection and identification according to the radio frequency data to generate myocardial mask; and estimating a large-displacement estimation term, a small-displacement estimation term, and a regularization term to construct a myocardial displacement estimation cost function; and obtaining the myocardial displacement parameters according to the cost function; and performing strain estimation. A myocardial multi-parametric ultrasound imaging system is further provided.

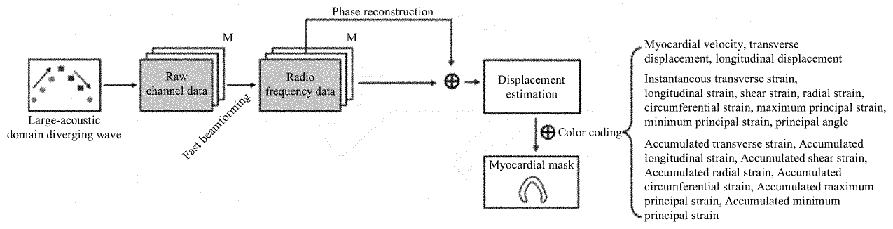

### CROSS-REFERENCE TO RELATED APPLICATIONS

This application claims the benefit of priority from Chinese Patent Application No. 202111677331.5, filed on Dec. 31, 2021, and Chinese Patent Application No. 202111677333.4, filed on Dec. 31, 2021. The content of the aforementioned application, including any intervening amendments thereto, is incorporated herein by reference in its entirety.

### TECHNICAL FIELD

This application relates to ultrasound imaging, particularly to high-frame rate multi-parametric ultrasound imaging techniques based on coherent compounding of diverging waves, and more particularly to a myocardial multiparametric ultrasound imaging method and system.

### BACKGROUND

As the technique with the highest frame rate in medical imaging, ultrasound imaging is also characterized by real-time detection and no radiation, and is applicable to the point-of-care testing (POCT). Currently, the ultrasound imaging has been widely used in cardiac structure and function imaging. Cardiac high-frame rate (HFR) imaging plays an important role in the cardiac monitoring. However, the traditional imaging methods fail to achieve the desirable cardiac tissue characterization due to rapid motion (with a heart rate of about 60 bpm under resting state and above 120 bpm under stress state).

Traditional echocardiography suffers the mutual constraint between frame rate and acoustic domain, in which the focused wave line-by-line scanning is employed for imaging. However, in order to cope with the rapid beating, the acoustic domain needs to be narrowed, and only all or part of the left ventricle can be imaged. In this case, the frame rate can reach about 70 Hz. In recent years, in order to overcome the above constraint, multi-beam scanning and small sector stitching methods have been used to obtain ultrasonic cardiograms with a large acoustic domain, and the frame rate can be improved to about 100 Hz. However, it is still unable to cope with the decorrelation noise caused by the low frame rate and rapid heart beating, which will cause great interference and error to the calculation and imaging of myocardial parameters (such as velocity and strain). In order to overcome the shortcomings of traditional methods, an echocardiographic imaging technique with high frame rate and large acoustic domain is urgently required.

The ultrafast plane-wave imaging technology proposed in recent years may be able to overcome the above shortcomings and satisfy the clinical needs. Theoretically, the plane wave imaging can reach a frame rate of more than 5000 Hz, which is enough to cope with the rapid cardiac pulsation (generally 60 bpm). However, due to the unfocused characteristic and low sound pressure emission of the plane wave, the signal-to-noise ratio of the obtained channel data is extremely low. French Tanter proposed a multi-angle coherent compounding technology to realize, at the expense of a certain frame rate, the high frame rate plane wave imaging with comparable quality to the focused wave imaging. However, due to the rib blocking, it is required to use the phased array to emit diverging waves, so as to realize the multi-angle high-frame rate coherent compounding imaging. However, there is a phase delay caused by the large inclination angle of the diverging wave and the rapid cardiac motion, which will cause misalignment between the scattering points of the same phase during the coherent compounding, resulting in phase shift interference. In particular, there is great side-lobe interference from the extremely low sound pressure zones on both sides of the large acoustic domain (such as four-chamber cardiac panoramic imaging), which makes the quality of the myocardial large acoustic domain imaging unsatisfactory and has great interference with the calculation and imaging of myocardial parameters (such as velocity and strain).

In addition, the high-precision multi-parametric imaging requires accurate estimation of rapid myocardial displacement changes during the cardiac cycle. Conventional estimation methods mainly include tissue Doppler estimation and cross-correlation estimation, which are suitable for the estimation of large displacements but are not sensitive to small myocardial displacements. Other methods, such as optical flow method, are sensitive to small displacement estimation, but are more sensitive to decorrelation noise, and thus are not suitable for the large displacement estimation. Therefore, how to balance the estimation of large displacement and small displacement in the myocardial displacement estimation to enhance the multiparametric imaging precision still remains to be urgently solved.

### SUMMARY

An object of this application is to provide a myocardial multi-parametric ultrasound imaging method and system to overcome the mutual constraint between frame rate and acoustic domain of the ultrasound imaging, unsatisfactory imaging quality, and great interference with myocardial parameters under the rapid cardiac pulsation, so as to better analyze the myocardial motion.

Technical solutions of this application are described as follows.

In a first aspect, this application provides a myocardial multi-parametric ultrasound imaging method, including the following steps (S1) to (S9).

(S1) A phased array probe alternately emits a set of M (M is an even number, such as M=32) large-acoustic domain diverging waves. The large-acoustic domain diverging waves include an ascending branch and a descending branch.

(S2) The corresponding M raw channel data Rᵢ (i=1, 2, . . . , N) is subjected to the beamforming by using the fast beamforming method to obtain the M beamforming radio frequency (RF) data Sⱼ (j=1, 2, . . . , M).

(S3) According to the M beamforming radio frequency data Sⱼ in an ascending branch and in a descending branch, an autocorrelation angle product is calculated to obtain a phase shift angle between the corresponding inclination angle caused by rapid myocardial motion, large inclination angle and large acoustic domain emission. Further, the radio frequency data are coherently compounded, while performing on phase shift compensation using the phase shift angle.

(S4) According to the phase shift compensation angle, Doppler velocity of each coherently-compounded radio frequency data is calculated to generate a Doppler velocity distribution threshold matrix at a moment, and myocardium at each moment of a cardiac cycle is segmented to obtain a myocardial mask. The Doppler velocity distribution within the myocardial mask is obtained.

(S5) A large-displacement estimation term, a small-displacement estimation term, and a regularization term are estimated to jointly construct a cost function of myocardial displacement estimation which is constrained by time-space domain to improve the robustness of the function.

(S6) Under a time-domain constraint and a space-domain constraint, weights of the large-displacement estimation term, the small-displacement estimation term, and the regularization term are iteratively and recursively updated to obtain an instantaneous myocardial displacement distribution.

(S7) According to the coherently-compounded radio frequency data with high frame rate and the myocardial mask, an instantaneous myocardial transverse displacement and an instantaneous myocardial longitudinal displacement are calculated. Myocardial strain is estimated including an instantaneous myocardial transverse strain, an instantaneous myocardial longitudinal strain, and an instantaneous myocardial shear strain.

(S8) According to the instantaneous myocardial transverse strain, the instantaneous myocardial longitudinal strain, and the instantaneous myocardial shear strain, an instantaneous myocardial radial strain, instantaneous myocardial circumferential strain and instantaneous myocardial principal component strain are calculated under different coordinate systems. At the same time, the instantaneous myocardial radial strain, the instantaneous myocardial circumferential strain and the instantaneous myocardial principal component strain are accumulated during the cardiac cycle to obtain the accumulated radial strain, accumulated circumferential strain and accumulated myocardial principal component strain.

(S9) The parametric distribution of the myocardial tissue, including myocardial velocity, displacement, instantaneous strain, and cumulative strain in the cardiac cycle, are performed with color-coded imaging, and high-frame rate and large-acoustic domain multi-parametric images of myocardial tissue can be reconstructed at one time.

In an embodiment, the step (S1) includes the following steps.

(S1.1) The ultrasonic phased array probe emits M large-acoustic domain diverging plane wave sequences which are the diverging wave sequences.

(S1.2) The diverging wave sequences are emitted at a vertical emission of 0°, and a virtual point is tilted left and right and changed at an inclination angle from −θ to +θ.

(S1.3) The inclination angles of the diverging wave sequences are divided into the ascending branch and the descending branch.

(S1.4) The inclination angles of 1 to M/2 sequences in the ascending branch increases from −θ to +θ-γ in uniform or non-uniform step γ.

(S1.5) The inclination angles of the M/2+1 to M sequences in the descending branch are reduced from +θ to −θ+γ in uniform or non-uniform step γ.

In an embodiment, the step (S2) includes the following steps.

(S2.1) According to the M large-acoustic domain diverging wave sequence with the inclination angles in the ascending branch or in the descending branch, the M raw channel data Rᵢ (i=1, 2, . . . , N) are obtained. Where N is the number of channels of the ultrasonic probe.

(S2.2) The beamforming is carried out according to the fast beamforming method to obtain the beamforming radio frequency data Sⱼ (j=1, 2, . . . , M). Fast beamforming using the delayed overlay method is described by the following formula:

$${s}_{j} ( t ) = {\sum }_{i = 1}^{N} {w}_{i} {R}_{i} ( t - {Δ}_{i} ) ,$$

where wᵢ represents the weight, and Δᵢ is the delay of the unit output.

In an embodiment, the step (S3) includes the following steps. (S3.1) The autocorrelation calculation of the radio frequency data Sⱼ (j=1, 2, . . . , M/2) obtained by beamforming of the diverging wave echo sequences in the ascending branch, the autocorrelation radio frequency data [symbol] ₁ of the ascending branch is obtained, and the calculation formula is expressed as:

$$( θ , γ ) = {\sum }_{j = 1}^{M / 2} \frac{{s}_{m} \overline{{s}_{m + 1}}}{\vert {s}_{m} \overline{{s}_{m + 1}} \vert} ,$$

where Sₘ is m-th radio frequency data at (θ, γ).

(S3.2) The autocorrelation calculation of the radio frequency data Sⱼ (j=M/2+1, M/2+2, . . . , M) obtained by beamforming of the diverging wave echo sequences in the descending branch, the autocorrelation radio frequency data [symbol] ₂ of the descending branch is obtained, and the calculation formula is expressed as:

$$( θ , γ ) = {\sum }_{j = \frac{M}{2} - 1}^{M} \frac{{s}_{m} \overline{{s}_{m + 1}}}{\vert {s}_{m} \overline{{s}_{m + 1}} \vert} .$$

(S3.3) According to the M radio frequency data Sⱼ (j=1, 2, . . . , M), the coherently-compounded radio frequency data \tilde{S}_{c} is obtained by adjusting the phase shift angle φ, the interframe displacement, and the phase rotation.

The phase shift angle resulting from the autocorrelation product is calculated by the following formula:

$$φ ( θ , γ ) = \frac{1}{2} ∠ \{ \}$$

The coherently-compounded radio frequency data is calculated by the following formula:

$${˜}^{} ( θ , γ ) == {\sum }_{m = 1}^{M} \{ {s}_{m} ( θ , r + ( m - \frac{M}{2} ) \frac{φ}{4 π} \frac{c}{{f}_{0}} ) {e}^{im φ} \} .$$

In an embodiment, the step (S4) includes the following steps.

(S4.1) The Doppler velocity of each coherently-compounded radio frequency data is calculated according to the phase shift compensation angle by the following formula:

$$tdi = \frac{PRFc}{4 π {f}_{0}} φ .$$

(S4.2) The Doppler velocity distribution threshold matrix at that moment is generated from the Doppler velocity distribution. The velocity distribution data greater than the threshold is set to 1, otherwise 0, so as to realize the automatic segmentation calculation of the myocardium for each frame of the cardiac cycle, and obtain the myocardial mask for each frame in the cardiac cycle.

(S4.3) The myocardial Doppler velocity distribution matrix is obtained by dot multiplying the myocardial mask of each frame in the cardiac cycle and the Doppler velocity distribution of the corresponding frame.

In an embodiment, the step (S5) includes the following steps.

(S5.1) Based on diverging wave sequence radio frequency data with the high-frame rate, the myocardial Doppler displacement distribution u is obtained by the Doppler calculation formula. Or from the radio frequency data of adjacent frames, the myocardial displacement distribution u is obtained by the cross-correlation estimation formula.

(S5.2) The Doppler displacement term or cross-correlation displacement term of myocardial tissue constitute the large-displacement estimation term J₁(u) of myocardial motion tracking.

(S5.3) Based on the optical flow method, the myocardial displacement distribution u between the radio frequency data of adjacent frames is obtained, which constitutes the small-displacement term J₂(u) of myocardial motion tracking.

(S5.4) The displacement u of the J₁ and J₂ terms is transformed into the Fourier domain, and the sum of squares of the divergence and curl constitutes the regularization term J_{reg}(u) of myocardial motion tracking.

(S5.5) The myocardial displacement estimation cost function J(u) is constructed from the large-displacement estimation term J₁, the small-displacement estimation term J₂ and the regularization term J_{reg}, and expressed as:

J(\vec{u})=αJ₁(\vec{u})+βJ₂(\vec{u})+δJ_{reg}(\vec{u}),

where α+β=1, which is an adjustment parameter used to characterize relative contribution of displacement, which can be verified by specific experiments.

(S5.6) The cost function J(u) is constrained by the myocardial mask Mk in the space domain and a short-time window T in the time domain, and expressed as follows:

J(\vec{u})=α∫∫_{Mk,T}J₁(\vec{u}ᵢ)+β∫∫_{Mk,T}J₂(\vec{u}ᵢ)+δ∫∫_{Mk,T}J_{reg}(\vec{u}ᵢ).

J₁ is the large-displacement estimation term. In this embodiment, J₁ is the Doppler term or the cross-correlation term. J₂ is the small-displacement estimation term. In this embodiment, J₂ is the optical flow term. Jreg is a regularization term. Mk is the integral local region determined by width of the half window of the myocardial segmentation region. Window T needs to be set to avoid out-of-plane movement.

In an embodiment, the optical flow term and the tissue Doppler term are expressed as follows:

$${J}_{of} ( {u}_{i} ) = {{ω}_{of} ( \overline{\vec{V} R} \cdot {\vec{u}}_{i} + \overline{{\partial }_{t} R} )}^{2}$$

$${J}_{tdi} ( {\vec{u}}_{i} ) = {{ω}_{tdi} ( {\vec{d}}_{tdi} \cdot {\vec{u}}_{i} - c \cdot PRF \cdot ϕ / 4 π {f}_{0} \cdot FR )}^{2} .$$

Among them, ω_{of} and ω_{tdi} are the weights of the optical flow term and the Doppler term, respectively. In the optical flow term, [symbol] is the normalized spatial gradient vector \vec{∇} [symbol] , and [symbol] is the normalized time gradient vector ∂ₜ [symbol] among the short-time dimension T. \vec{∇} [symbol] ={∂_{θ} [symbol] ∂ᵣ [symbol] } is the gradient vector in the radial and angular directions of ultra-fast composite divergent echocardiogram. In the Doppler term, \vec{d}_{tdi}={∂_{θ}d_{tdi}∂ₜd_{tdi}} shows the unit displacement vector ∂_{θ}d_{tdi} and short-time scales ∂ₜd_{tdi} in the Doppler direction. c, PRF, ϕ, f₀ and FR respectively represent the speed of sound in the tissue, the pulse repetition rate, the phase shift angle between transmissions, the pulse emission frequency and the frame rate after coherently-compounded.

In an embodiment, the regularization term is rewritten in the frequency domain and simplified to the following formula:

J_{reg}(\vec{U}ᵢ)=∥\vec{ξ}ᵢ∥²[(\vec{ξ}ᵢ·\vec{ũ}ₗ)²+(\vec{ξ}ᵢ×\vec{ũ}ₗ)²]≈∥\vec{ξ}ᵢ∥⁴∥\vec{ũ}₁∥²,

where \vec{ξ}ᵢ is the spatial Fourier frequency of \vec{u}ᵢ. When δ in the regularization term is defined as (1/ξ_{c})⁴, the displacement estimation model is rewritten as the following formula:

$$J ( \vec{u} ) = \{ \begin{matrix}α \int {\int }_{Mk , T} {J}_{of} ( {\vec{u}}_{i} ) + β \int {\int }_{Mk , T} {J}_{tdi} ( {\vec{u}}_{i} ) & \vert \vec{ξ} \vert ≪ {ξ}_{c} \\ α \int {\int }_{Mk , T} {J}_{of} ( {\vec{u}}_{i} ) + β \int {\int }_{Mk , T} {J}_{tdi} ( {\vec{u}}_{i} ) + δ \int {\int }_{Mk , T} {J}_{reg} ( {\vec{u}}_{i} ) & others \\ 0 & \vert \vec{ξ} \vert ≫ {ξ}_{c}\end{matrix} ,$$

where ξ_{c} is spatial cutoff frequency.

In an embodiment, the step (S6) includes the following steps.

(S6.1) The weights of the large-displacement estimation term, the small-displacement estimation terms, and the regularization term are iteratively updated.

(S6.2) The weights of the large-displacement estimation term, the small-displacement estimation terms, and regularization term are recursively updated.

In an embodiment, the motion estimation model estimates the adjustment parameters α, β and δ of instantaneous myocardial displacement, and the model is solved as an optimization problem to minimize the quadratic short-time spatial cost function in the iterative process. The bisquare function H(·) updates the weights of ω_{of} and ω_{tdi} point by point and assigns smaller weights to the small-displacement term (the optical flow term) and the larger residuals point in the large-displacement term (the tissue Doppler term or the cross-correlation term) respectively, and H(·) is expressed as follows:

ω_{of}ⁱ⁺¹=ω₀·H[J_{of}(\vec{U}ᵢ)/ω_{of}ⁱ]

ω_{tdi}ⁱ⁺¹=ω₀·H[J_{tdi}(\vec{U}ᵢ)/ω_{tdi}ⁱ]

ω₀=4V_{N}/|σ_{D}+2V_{N}−FR|,

where ω₀∈[0, 1] is the initialization value of ω_{of} and ω_{tdi} during iteration, and i is the number of iterations. ω₀∈[0, 2V_{N}] is the Doppler standard deviation, and V_{N} is the Nyquist Doppler velocity.

(S6.3) An instantaneous myocardial displacement distribution within the short-time window T is obtained. Another instantaneous myocardial displacement distribution in next short-time window T is determined, the short-time window has certain overlapping rate, the instantaneous myocardial displacement distribution in the next short-term window T is obtained repetitively. The K-frame radio frequency data is completely traversed to obtain the instantaneous myocardial displacement distribution with high frame rate.

In an embodiment, the step (S7) includes the following steps.

According to the instantaneous myocardial transverse displacement and the instantaneous myocardial longitudinal displacement, a two-dimensional strain estimator is provided to estimate the myocardial strain to obtain the instantaneous myocardial transverse strain, the instantaneous myocardial longitudinal strain, and the instantaneous myocardial shear strain.

In an embodiment, the step (S8) includes the following steps.

(S8.1) According to the instantaneous myocardial transverse strain, the instantaneous myocardial longitudinal strain, and the instantaneous myocardial shear strain, a geometric center of a target area is determined. The instantaneous myocardial radial strain and instantaneous myocardial circumferential strain with the geometric center as a coordinate origin are obtained under the polar coordinate system.

(S8.2) According to the instantaneous myocardial transverse strain, the instantaneous myocardial longitudinal strain, and the instantaneous myocardial shear strain, a principal component analysis is carried out to obtain a maximum instantaneous myocardial principal strain, a minimum instantaneous myocardial principal strain, an angle distribution between a maximum and a minimum principal component under a principal coordinate system.

(S8.3) According to the above instantaneous strain, an accumulation is carried out in the cardiac cycle to obtain an accumulated radial strain, an accumulated circumferential strain, an accumulated maximum principal strain, and an accumulated minimum principal strain.

In an embodiment, the step (S9) includes the following steps.

(S9.1) Taking the zero point in each frame of myocardial mask as the reference position, the corresponding positions of the myocardial velocity, the transverse displacement, and the longitudinal displacement distribution are replaced as null values, and then the non-null regions perform color-coding to obtain high-frame-rate large-acoustic domain myocardial tissue images of velocity, transverse displacement, and longitudinal displacement.

(S9.2) Taking the zero point in each frame of myocardial mask as the reference position, the instantaneous transverse strain, longitudinal strain, shear strain, radial strain, circumferential strain, maximum principal strain, minimum principal strain, and corresponding position of the auxiliary parameter of the principal angle are placed as null values, and the non-null regions perform color-coding to obtain high-frame-rate large-acoustic domain myocardial tissue auxiliary parametric images of transverse strain, longitudinal strain, shear strain, radial strain, circumferential strain, maximum principal strain, minimum instantaneous myocardial principal strain and the angle between the principal components.

(S9.3) Taking the zero point in each frame of myocardial mask as the reference position, the cumulative transverse strain, longitudinal strain, shear strain, radial strain, circumferential strain, maximum principal strain, and minimum principal strain in the cardiac cycle are replaced with null values, and the non-null regions perform color-coding to obtain high-frame-rate large-acoustic domain myocardial tissue cumulative strain images, including cumulative transverse strain, cumulative longitudinal strain, cumulative shear strain, cumulative radial strain, cumulative circumferential strain, cumulative maximum principal strain, and cumulative minimum principal strain.

The diverging wave with a beam width greater than 60° on the heart is considered as large acoustic domain.

Compared to the prior art, this application has the following beneficial effects.

A myocardial multi-parametric ultrasound imaging method emits alternately a plurality of the ascending and descending large-acoustic domain diverging waves into myocardial tissue to obtain a plurality of raw channel data. The myocardial multi-parametric ultrasound imaging method performs a beamforming using a fast beamforming method to obtain a plurality of radio frequency data. According to the radio frequency data, imaging parameters are reconstructed. The myocardial multi-parametric ultrasound imaging method overcomes the choice between frame rate and acoustic domain of ultrasound imaging, non-ideal imaging quality, and great interference on myocardial tissue parameters in rapid heartbeat, and improves coherence recombination of sidelobe artifacts and phase delay interference caused by large angles when the tissue is rapidly deformed. The myocardial multi-parametric ultrasound imaging method eliminates the phase delay caused by rotation, retains the phase delay caused by tissue movement, combines alternating sequences and autocorrelation products, and improves the contrast and resolution of cardiac images. The myocardial multi-parametric ultrasound imaging method can obtain high-frame-rate large-acoustic domain myocardial velocity, displacement, instantaneous strain, and accumulated strain images of up to 20 parameters.

Two autocorrelated angular products are used to estimate the transmission phase delay caused by motion, and the phase delay of the correspond to tissue movement, and the phase delays are used to perform phase reconstruct on the beamforming data to obtain myocardial masks. The large-displacement estimation term, the small-displacement estimation term, and regularization term are estimated to construct the cost function of myocardial displacement estimation, balance the displacement term, realize the accurate estimation of myocardial motion during cardiac rapid fluctuations, and obtain a coherently-compounded diverging echocardiogram with high contrast and resolution under the stress of motion compensation.

### BRIEF DESCRIPTION OF THE DRAWINGS

This FIGURE is a flow chart of a myocardial multi-parametric ultrasound imaging method according to one embodiment of the present disclosure.

### DETAILED DESCRIPTION OF EMBODIMENTS

The technical solutions of the disclosure will be described in detail below with reference to the drawings and embodiments to make those skilled in the art understand the technical solutions better. Described below are merely preferred embodiments of the disclosure, which are not intended to limit the disclosure. For those skilled in the art, other embodiments obtained based on these embodiments without paying creative efforts should fall within the scope of the disclosure defined by the appended claims.

It should be noted that the terms “first” and “second” are used to distinguish similar objects and cannot be understood as a particular order or sequence. It should be understood that the terms may be interchangeable where appropriate, so that the embodiments described herein may be embodied in other orders not described herein. Further, the terms “comprising” and “having”, and any variation thereof are intended to cover non-exclusive inclusions. For example, a process, method, system, product or equipment comprising a series of steps or units need not be limited to those steps or units listed, but may include other steps or units not clearly listed or inherent to the process, method, product or equipment.

A myocardial multi-parametric ultrasound imaging method provided in the disclosure includes the generation and reception of alternating emission sequences of large-acoustic domain diverging waves, the calculation of autocorrelation angle product, the adjustment of inter-frame displacement and phase rotation, the combination of small displacement tracking and large displacement tracking, the multi-parametric imaging of strain calculation using instantaneous displacement. In cardiac ultrasound imaging, the high-frame-rate large-acoustic domain myocardial multi-parametric ultrasound imaging method achieves high spatiotemporal resolution, contrast, motion estimation accuracy and obtains a variety of parameters for imaging. The imaging method is central to the autocorrelation motion phase shift compensation of the ascending and descending alternating transmission sequence. Among them, the range of large acoustic domain ≤120°, and the range of high frame rate ≤5000 Hz.

In this embodiment, an alternating diverging wave sequence includes rising and falling stages. M coherent raw channel data Rᵢ is acquired and performs beamforming with delayed superposition to obtain beamforming radio frequency data Sⱼ. The autocorrelation of ascending and descending branches using M beamforming Radio frequency data is calculated. For a given length M, two autocorrelations along the time axis are calculated, and the two autocorrelations correspond to the rising and falling stages of the diverging wave sequences, respectively. The product of two autocorrelation angles is used to estimate the transmission phase delay caused by motion and the phase delay of the corresponds to tissue movement. These phase delays are used to perform phase reconstruction on the beamforming radio frequency data to obtain the high-frame-rate coherently-compounded diverging echocardiogram with high contrast and resolution under the stress of motion compensation. Myocardial masks are obtained, and the cost function of myocardial displacement estimation is constructed by large-displacement estimation terms, small-displacement estimation terms and regularization terms, and the robustness of the function is improved by time domain and spatial domain constraints. The numerical optimization and iterative solution models are used to obtain high-frame-rate cardiac ultrasound myocardial displacement estimation. On the basis of displacement estimation, the strain estimation is carried out, and multi-parameter myocardial imaging is performed.

Referring to an embodiment shown in the FIGURE, the myocardial multi-parametric ultrasound imaging method includes the following steps.

(1) A set of M (M is an even number, such as M=32) large-acoustic domain diverging planar wave sequences are alternately emitted. The large-acoustic domain diverging planar wave sequences include an ascending branch and a descending branch Fast beamforming is carried out using the received raw channel data.

(2) According to the obtained beamforming radio frequency data, the autocorrelation radio frequency data [symbol] ₁, [symbol] ₂ of the ascending and descending branches are calculated respectively, and the autocorrelation angle product between the two branches are calculated. The autocorrelation radio frequency data for the ascending and descending branches are represented by the following formula, respectively:

$$( θ , γ ) = {\sum }_{j = 1}^{M / 2} \frac{{s}_{m} \overline{{s}_{m + 1}}}{\vert {s}_{m} \overline{{s}_{m + 1}} \vert}$$

$$( θ , γ ) = {\sum }_{j = \frac{M}{2} - 1}^{M} \frac{{s}_{m} \overline{{s}_{m + 1}}}{\vert {s}_{m} \overline{{s}_{m + 1}} \vert} ,$$

where Sₘ is m-th radio frequency data at (θ, γ). There are j groups radio frequency data in all, which is composed of ascending branch (j=1, 2, . . . , M/2) and descending branch (j=M/2+1, M/2+2, . . . , M).

The phase shift angle φ resulting from the product of autocorrelation angles is calculated according to the following formula:

$$φ ( θ , γ ) = \frac{1}{2} ∠ \{ \} .$$

According to the phase shift angle φ, the interframe displacement and the phase rotation are adjusted to obtain the coherently-compounded radio frequency data \tilde{S}_{c} of M radio frequency data Sⱼ (j=1, 2, . . . , M).

The coherently-compounded radio frequency data is calculated by the following formula:

$${˜}^{} ( θ , γ ) == {\sum }_{m = 1}^{M} \{ {s}_{m} ( θ , r + ( m - \frac{M}{2} ) \frac{φ}{4 π} \frac{c}{{f}_{0}} ) {e}^{im φ} \} .$$

(3) The Doppler velocity of each coherently-compounded radio frequency data is calculated to generate the distribution threshold matrix, and myocardial masks are obtained by myocardial segmentation calculation.

The Doppler velocity is calculated by the following formula:

$$tdi = \frac{PRFc}{4 π {f}_{0}} ϕ .$$

(4) Based on coherently-compounded myocardial motion radio frequency data with the high frame rate, the Doppler displacement distribution u of myocardial tissue is obtained by the Doppler calculation formula. Or from the radio frequency data of adjacent frames, the myocardial displacement distribution u is obtained by the cross-correlation estimation formula.

(5) Based on the corresponding estimation method, the large-displacement estimation term J₁ and the small-displacement estimation term J₂ are constructed, and the displacement is transformed in the Fourier domain. The regularization term J_{reg}(u) of myocardial motion tracking is constituted by the sum of squares of divergence and curl. The cost function J(u) of the myocardial displacement estimation is constructed from the large-displacement estimation term J₁, the small-displacement estimation term J₂, and the regularization term J_{reg} and expressed as:

J(\vec{u})=αJ₁(\vec{u})+βJ₂(\vec{u})+δJ_{reg}(\vec{u});

where α+β=1, which is an adjustment parameter used to characterize relative contribution of displacement, which can be verified by specific experiments. The cost function J(u) is constrained by the myocardial mask Mk in the space domain and the short-time window Tin the time domain, and expressed as follows:

J(\vec{u})=α∫∫_{Mk,T}J₁(\vec{u}ᵢ)+β∫∫_{Mk,T}J₂(\vec{u}ᵢ)+δ∫∫_{Mk,T}J_{reg}(\vec{u}ᵢ)

(6) The weights of the large-displacement estimation term, the small-displacement estimation terms, and the regularization term are iteratively and recursively updated.

The motion estimation model estimates the adjustment parameters (α, β and δ) of instantaneous myocardial displacement and solves the model as an optimization problem to minimize the quadratic short-time spatial cost function in the iterative process. The instantaneous myocardial displacement distribution within the short-time window T is obtained. Another instantaneous myocardial displacement distribution in next short-time window T is determined, the short-time window has a certain overlapping rate, the instantaneous myocardial displacement distribution in the next short-term window T is obtained repetitively. The K-frame radio frequency data is completely traversed to obtain the instantaneous myocardial displacement distribution with high frame rate. Then, the two-dimensional strain estimator is used to estimate the myocardial strain, and the transverse strain, longitudinal strain, and shear strain are obtained.

(7) The instantaneous myocardial radial strain and instantaneous myocardial circumferential strain is obtained under a polar coordinate system based on the previous step, the instantaneous myocardial transverse strain, the instantaneous myocardial longitudinal strain, and the instantaneous myocardial shear strain are obtained in the previous step. The maximum and minimum instantaneous myocardial principal strains, as well as the maximum and minimum principal angle distributions are obtained under the principal coordinate system. The accumulated radial strain, accumulated circumferential strain, and accumulated maximum and minimum principal strains are calculated according to the instantaneous parameters.

(8) The parametric distribution of myocardial tissue generated in the previous step is performed with color-coded imaging. High-frame-rate large-acoustic domain myocardial tissue auxiliary parametric images of velocity, transverse displacement, and longitudinal displacement are obtained by reconstruction. Images of transverse strain, longitudinal strain, shear strain, radial strain, circumferential strain, maximum principal strain, minimum instantaneous myocardial principal strain and images of the angle between the principal strain are obtained. Images of cumulative transverse strain, cumulative longitudinal strain, cumulative shear strain, cumulative radial strain, cumulative circumferential strain, cumulative maximum principal strain, and cumulative minimum principal strain are obtained.

In this embodiment, the myocardial multi-parametric ultrasound imaging system includes an emission and acquisition module, a preprocessing module, and an imaging module.

The emission and acquisition module is configured to emit alternately a plurality of the ascending and descending large-acoustic domain diverging waves into myocardial tissue to obtain a plurality of raw channel data, and perform the beamforming using a fast beamforming method to obtain a plurality of beamforming radio frequency data.

The preprocessing module is configured to calculate the autocorrelation angle product of the radio frequency data of the ascending and descending large-acoustic domain diverging waves. The preprocessing module is configured to obtain a phase shift angle, caused by a rapid myocardial motion, a large inclination angle)(<30° and a large acoustic domain emission, between the inclination angles in the ascending branch and in the descending branch. The preprocessing module is configured to coherently compound the radio frequency data of inclination angles in the ascending branch and in the descending branch, and simultaneously perform phase shift compensation using the phase shift angle.

According to a phase shift compensation angle, Doppler velocity of each coherently-compounded radio frequency data is calculated, the Doppler velocity distribution threshold matrix at a moment is generated, and myocardium at each moment of the cardiac cycle is segmented to obtain the myocardial mask.

According to the high-frame-rate coherently-compounded radio frequency data and the myocardial mask, the large-displacement estimation term, the small-displacement estimation term, and the regularization term are obtained to jointly construct the myocardial displacement estimation cost function.

Under a time-domain constraint and a space-domain constraint, weights of the large-displacement estimation term, the small-displacement estimation term, and the regularization term are iteratively and recursively updated to obtain the instantaneous myocardial displacement distribution.

According to the instantaneous myocardial transverse displacement and an instantaneous myocardial longitudinal displacement of the myocardium, the myocardial strain is estimated to obtain an instantaneous myocardial transverse strain, an instantaneous myocardial longitudinal strain, and an instantaneous myocardial shear strain.

According to the instantaneous myocardial transverse strain, the instantaneous myocardial longitudinal strain, and the instantaneous myocardial shear strain, the instantaneous myocardial radial strain, a circumferential strain and an instantaneous myocardial principal strain are reconstructed under different coordinate systems.

The imaging module is configured to perform color-coded imaging, according to the instantaneous myocardial transverse strain, the instantaneous myocardial longitudinal strain, the instantaneous myocardial shear strain, the instantaneous myocardial radial strain, the instantaneous myocardial circumferential strain and the instantaneous myocardial principal strain processed by the preprocessing module, to reconstruct and obtain multi-parametric images of myocardial tissue.

In this embodiment, a terminal device includes a processor and a memory. The memory is used to store a computer program. The computer program includes program instructions, and the processor is used to execute the program instructions stored by the computer storage medium. The processor adopts a central processing unit (CPU), or adopts other general-purpose processors, digital signal processors (DSPs), application-specific integrated circuits (ASICs), field programmable gate arrays (FPGAs) or other programmable logic devices, discrete gates or transistor logic devices, and discrete hardware components, which are the computing core and control core of the terminal and suitable for implementing one or more instructions. Specifically, the processor described is suitable for loading and executing one or more instructions to achieve the corresponding method flow or corresponding functions. The processor described in this embodiment may be used for executing the multi-parametric ultrasound imaging method.

In this embodiment, a storage medium uses a computer-readable storage medium. The computer-readable storage medium is a memory device in the terminal device, for storing programs and data. The computer-readable storage medium includes a built-in storage medium in the terminal device, providing storage space, storing the operating system of the terminal, and may also include an extended storage medium supported by the terminal device. Further, the storage space also stores one or more instructions suitable for being loaded and executed by the processor, and the instructions may be one or more computer programs (including program code). Specifically, the computer-readable storage medium may be high-speed RAM memory or non-volatile memory, such as at least one disk memory. One or more instructions stored in a computer-readable storage medium may be loaded and executed by the processor to execute the multi-parametric ultrasound imaging method.

The myocardial multi-parametric ultrasound imaging method has the following advantages.

1. The method overcomes the choice between frame rate and acoustic domain of ultrasound imaging during rapid tissue movement and deformation in the traditional echocardiography, and overcomes the sidelobe artifacts and phase delay interference caused by large angles, improving the contrast and resolution of ultrasonic cardiogram.

2. Large-displacement estimation and small-displacement estimation are balanced to overcome the problems of decorrelation noise and out-of-plane motion. The method provides accurate and objective two-dimensional myocardial strain assessment and motion displacement estimation in clinical practice using high heart rate echocardiography, which improves the sensitivity and contrast of complex and rapid myocardial deformation displacement estimation methods.

3. One-time reconstruction of high-frame-rate and large-acoustic domain ultrasound myocardial tissue parametric imaging is up to 20 parameters, providing a reliable criterion for ultrasound detection of cardiac moving tissues.

4. The method with high frame rate and large acoustic domain can be applied to more accurate, stable and robust strain estimators to improve the accuracy of cardiac parameter detection.

The method in this disclosure first emits the large-acoustic domain diverging wave sequences with the ascending and descending inclination angles to obtain a plurality of phase-coherent receiving echoes, followed by beamforming. The obtained beamforming signals are performed with phase shift rotation and compensation by the autocorrelation angle product of different inclination angles to obtain high-frame-rate coherently-compounded myocardial radio frequency data, reduce sidelobe and phase shift interference. The large displacement and the small displacement are combined to provide a framework for myocardial motion displacement estimation and strain estimation, and obtain high-frame-rate large-acoustic domain multi-parametric echocardiogram including instantaneous strain and cumulative strain parameters.

What is claimed is:

1. A myocardial multi-parametric ultrasound imaging method, comprising:
(S1) emitting alternately a plurality of large-acoustic domain diverging waves to myocardial tissue to obtain a plurality of raw channel data, wherein the plurality of large-acoustic domain diverging waves comprise an ascending branch and a descending branch; and subjecting the plurality of raw channel data to beamforming by using a fast beamforming method to obtain a plurality of beamforming radio frequency data;
(S2) calculating an autocorrelation angle product between radio frequency data in an ascending branch and radio frequency data in a descending branch; obtaining a phase shift angle between corresponding inclination angles, wherein the phase shift angle is caused by rapid myocardial motion, large inclination angle and large acoustic domain emission; and subjecting the plurality of radio frequency data to coherent compounding to obtain a plurality of coherently-compounded radio frequency data and simultaneously performing phase shift compensation using the phase shift angle;
(S3) according to a phase shift compensation angle, calculating a Doppler velocity of each of the coherently-compounded radio frequency data to generate a Doppler velocity distribution threshold matrix at a moment, and segmenting myocardium at each moment of a cardiac cycle to obtain a myocardial mask;
(S4) according to the coherently-compounded radio frequency data and the myocardial mask, obtaining a large-displacement estimation term, a small-displacement estimation term, and a regularization term to construct a myocardial displacement estimation cost function;
(S5) under a time-domain constraint and a space-domain constraint, iteratively and recursively updating weights of the large-displacement estimation term, the small-displacement estimation term, and the regularization term to obtain an instantaneous myocardial displacement distribution;
(S6) according to the coherently-compounded radio frequency data and the myocardial mask, calculating an instantaneous myocardial transverse displacement and an instantaneous myocardial longitudinal displacement, and estimating myocardial strain to obtain an instantaneous myocardial transverse strain, an instantaneous myocardial longitudinal strain, and an instantaneous myocardial shear strain;
(S7) obtaining an instantaneous myocardial radial strain, an instantaneous myocardial circumferential strain and an instantaneous myocardial principal strain under different coordinate systems according to the instantaneous myocardial transverse strain, the instantaneous myocardial longitudinal strain, and the instantaneous myocardial shear strain; and
(S8) according to the instantaneous myocardial transverse strain, the instantaneous myocardial longitudinal strain, the instantaneous myocardial shear strain, the instantaneous myocardial radial strain, the instantaneous myocardial circumferential strain and the instantaneous myocardial principal strain, performing color-coded imaging to obtain a myocardial multi-parametric image.

2. The myocardial multi-parametric ultrasound imaging method of claim 1, wherein the plurality of the large-acoustic domain diverging waves are emitted alternately by a phased array probe.

3. The myocardial multi-parametric ultrasound imaging method of claim 1, wherein the plurality of large-acoustic domain diverging waves are emitted alternately by an ultrasound probe to the myocardial tissue under a high-speed beat.

4. The myocardial multi-parametric ultrasound imaging method of claim 3, wherein the myocardial tissue has a resting state and a stress test state; and a heart rate of the resting state is 55-75 bpm, and a heart rate of the stress test state is equal to or greater than 120 bpm.

5. The myocardial multi-parametric ultrasound imaging method of claim 2, wherein the plurality of the large-acoustic domain diverging waves emitted by the phased array probe are large-acoustic domain diverging plane wave sequences.

6. The myocardial multi-parametric ultrasound imaging method of claim 1, wherein the plurality of radio frequency data Sⱼ (j=1, 2, . . . , M/2) in an ascending branch are subjected to autocorrelation calculation to obtain autocorrelated radio frequency data [symbol] ₁;
the plurality of radio frequency data Sⱼ (j=M/2+1, M/2+2, . . . , M) in a descending branch are subjected to autocorrelation calculation to obtain autocorrelated radio frequency data [symbol] ₂; and
the plurality of coherently-compounded radio frequency data \tilde{S}_{c} are obtained from M radio frequency data Sⱼ (j=1, 2, . . . , M) by adjusting interframe displacement and phase rotation according to the phase shift angle φ.

7. The myocardial multi-parametric ultrasound imaging method of claim 1, wherein in step (S3), in the Doppler velocity distribution threshold matrix, a velocity distribution data greater than a threshold is set to 1, otherwise 0, so as to obtain the myocardial mask for each frame in the cardiac cycle; and
a myocardial Doppler velocity distribution matrix is obtained by dot multiplying the myocardial mask of each frame in a cardiac cycle and doppler velocity distribution of a corresponding frame.

8. The myocardial multi-parametric ultrasound imaging method of claim 1, wherein the myocardial displacement estimation cost function J(u) is expressed as:
J(\vec{u})=αJ₁(\vec{u})+βJ₂(\vec{u})+δJ_{reg}(\vec{u});
wherein J₁ represents the large-displacement estimation term; J₂ represents the small-displacement estimation term; and J_{reg} represents the regularization term; α+β=1; the large-displacement estimation term J₁ comprises a Doppler term and a cross-correlation term; the small-displacement estimation term J₂ comprises an optical flow term; the regularization term Jreg comprises a one-norm regularization term, a two-norm regularization term, and a mask-constrained regularization term;
the myocardial displacement estimation cost function J(u) is constrained by the myocardial mask Mk in space domain and a short-time window T in time domain, expressed as follows:
J(\vec{u})=α∫∫_{Mk,T}J₁(\vec{u}ᵢ)+β∫∫_{Mk,T}J₂(\vec{u}ᵢ)+δ∫∫_{Mk,T}J_{reg}(\vec{u}ᵢ).

9. The myocardial multi-parametric ultrasound imaging method of claim 1, wherein the weights of the large-displacement estimation term, the small-displacement estimation terms, and the regularization term are iteratively updated; and the weights of the large-displacement estimation term, the small-displacement estimation terms, and regularization term are recursively updated.

10. The myocardial multi-parametric ultrasound imaging method of claim 8, wherein an instantaneous myocardial displacement distribution within the short-time window T is obtained; another instantaneous myocardial displacement distribution in next short-time window T is determined, and the short-time window T has a certain overlapping rate, the weights are iteratively and recursively updated to obtain an instantaneous myocardial displacement distribution in the next short-term window T; and K-frame radio frequency data is completely traversed to obtain the instantaneous myocardial displacement distribution.

11. The myocardial multi-parametric ultrasound imaging method of claim 10, wherein according to the instantaneous myocardial transverse displacement and the instantaneous myocardial longitudinal displacement, the myocardial strain is estimated by using a two-dimensional strain estimator to obtain the instantaneous myocardial transverse strain, the instantaneous myocardial longitudinal strain, and the instantaneous myocardial shear strain.

12. The myocardial multi-parametric ultrasound imaging method of claim 11, wherein according to the instantaneous myocardial transverse strain, the instantaneous myocardial longitudinal strain, and the instantaneous myocardial shear strain, a geometric center of a target area is determined; and the instantaneous myocardial radial strain and instantaneous myocardial circumferential strain with the geometric center as a coordinate origin are obtained under a polar coordinate system.

13. The myocardial multi-parametric ultrasound imaging method of claim 12, wherein according to the instantaneous myocardial transverse strain, the instantaneous myocardial longitudinal strain, and the instantaneous myocardial shear strain, a principal component analysis is carried out to obtain a maximum instantaneous myocardial principal strain, a minimum instantaneous myocardial principal strain, an angle distribution between a maximum principal component and a minimum principal component under a principal coordinate system.

14. The myocardial multi-parametric ultrasound imaging method of claim 13, wherein an accumulated radial strain, an accumulated circumferential strain, an accumulated maximum principal strain, and an accumulated minimum principal strain are obtained through accumulation in the cardiac cycle.

15. A myocardial multi-parametric ultrasound imaging system, comprising:
an emission and acquisition module;
a preprocessing module; and
an imaging module;
wherein the emission and acquisition module is configured to emit alternately a plurality of large-acoustic domain diverging waves in an ascending branch and a descending branch, respectively, obtain a plurality of raw channel data, and perform beamforming using a fast beamforming method to obtain a plurality of beamforming radio frequency data.
the preprocessing module is configured to calculate an autocorrelation angle product of the plurality of radio frequency data of the ascending and descending large-acoustic domain diverging waves; obtain a phase shift angle caused by rapid myocardial motion, large inclination angle and large acoustic domain emission; coherently compound the plurality of radio frequency data to obtain a plurality of coherently-compounded radio frequency data, and simultaneously perform phase shift compensation using the phase shift angle; calculate a Doppler velocity of each of coherently-compounded radio frequency data to generate a Doppler velocity distribution threshold matrix, perform segmented calculation on myocardium at each moment of a cardiac cycle to obtain a myocardial mask; obtain a large-displacement estimation term, a small-displacement estimation term, and a regularization term to construct a myocardial displacement estimation cost function according to the coherently-compounded radio frequency data and the myocardial mask; iteratively and recursively update weights of the large-displacement estimation term, the small-displacement estimation term, and the regularization term to obtain an instantaneous myocardial displacement distribution under a time-domain constraint and a space-domain constraint; calculate an instantaneous myocardial transverse displacement and an instantaneous myocardial longitudinal displacement according to the instantaneous myocardial displacement distribution; estimate myocardial strain to obtain an instantaneous myocardial transverse strain, an instantaneous myocardial longitudinal strain, and an instantaneous myocardial shear strain; and obtain an instantaneous myocardial radial strain, an instantaneous myocardial circumferential strain and an instantaneous myocardial principal strain through reconstruction under different coordinate systems; and
the imaging module is configured to perform color-coded imaging, according to the instantaneous myocardial transverse strain, the instantaneous myocardial longitudinal strain, the instantaneous myocardial shear strain, the instantaneous myocardial radial strain, the instantaneous circumferential myocardial strain and the instantaneous myocardial principal strain obtained by the preprocessing module to obtain a myocardial multi-parametric image.
